(19) United States
(12) Patent Application Publication
(10) Pub. No.: US 2026/0232579 A1
(43) Pub. Date: Aug. 13, 2026

(54) ORAL MUCOSAL FORMULATIONS OF ASPIRIN

(71) Applicant: Aspire Biopharma, Inc., Humacao, PR (US)

(72) Inventors: James Dzandu, Phoenix, AZ (US); Zafar Iqbal, Ramsey, NJ (US)

(21) Appl. No.: 19/471,300

(22) PCT Filed: Mar. 29, 2024
(86) PCT No.: PCT/US2024/022318

§ 371 Date: Sep. 30, 2025

Related U.S. Application Data
(60) Provisional application No. 63/456,290, filed on Mar. 31, 2023.

(51) Int. Cl. A61K 9/00; A61K 9/14; A61K 31/616; A61P 29/00

(52) U.S. Cl. CPC A61K 9/006; A61K 9/14; A61K 31/616; A61P 29/00

(57) ABSTRACT

Disclosed herein, in part, are pharmaceutical formulations for oral mucosal (e.g., sublingual and/or buccal) administration comprising micronized aspirin, (e.g., 70 mg to 90 mg micronized aspirin), a buffer, a surfactant, and one or more pharmaceutically acceptable excipients, wherein the pH of a solution of the formulation dissolved in 1 mL distilled water is less than 3. Methods of treating a subject in need thereof comprising administering a disclosed pharmaceutical formulation are also provided herein.

### CROSS REFERENCE TO RELATED APPLICATIONS

This application claims the benefit of and priority to U.S. Provisional Patent Application No. 63/456,290, filed on Mar. 31, 2023, the disclosure of which is hereby incorporated by reference in its entirety for all purposes.

### BACKGROUND

Aspirin is an established drug that has been in common use around the world for decades. Aspirin is a non-steroidal anti-inflammatory drug (NSAID) that is useful in treating and/or reducing many pathologies, including pain, fever, and inflammation. A patient suffering from a condition treatable with aspirin may benefit from the rapid delivery of aspirin, and there is thus a need to develop pharmaceutical formulations and/or dosage forms that allow for rapid absorption of aspirin into a patient's circulatory system.

### SUMMARY

Described herein are compositions, pharmaceutical formulations, and dosage forms useful for delivering aspirin to a patient in need thereof, e.g., a patient suffering from a disease or disorder treatable with aspirin, as described herein. Also provided herein are methods of treating a patient suffering from a disease or disorder described herein, the methods comprising administering to the subject a formulation described herein. Also described herein are methods for making pharmaceutical formulations described herein.

In one aspect, provided herein is a pharmaceutical formulation for oral mucosal (e.g., sublingual and/or buccal) administration comprising:
70 mg to 90 mg micronized aspirin;
a buffer;
a surfactant;
and one or more pharmaceutically acceptable excipients,
wherein the pH of a solution of the formulation dissolved in 1 mL distilled water is less than or equal to 3.

In another aspect, provided herein is a pharmaceutical formulation for oral mucosal (e.g., sublingual and/or buccal) administration comprising:
70 mg to 90 mg micronized aspirin;
56 mg to 75 mg citric acid anhydrous;
1 mg to 5 mg tri-potassium citrate monohydrate;
1 mg to 10 mg sodium lauryl sulfate; and
one or more pharmaceutically acceptable excipients.

In another aspect, provided herein is a pharmaceutical formulation for oral mucosal (e.g., sublingual and/or buccal) administration comprising:
81 mg micronized aspirin;
64 mg citric acid anhydrous;
2.4 mg tri-potassium citrate monohydrate;
6.8 mg sodium lauryl sulfate; and
one or more pharmaceutically acceptable excipients.

In another aspect, provided herein is a kit for suitable storage of an oral mucosal (e.g., sublingual and/or buccal) aspirin formulation comprising:
a pouch suitable for packaging a formulation disclosed herein; and
a formulation disclosed herein.

In another aspect, provided herein is a method of treating a disease, disorder, or condition in a subject in need thereof, the method comprising administering to the subject a formulation disclosed herein.

### BRIEF DESCRIPTION OF THE DRAWINGS

FIG. 1 is a series of microscopic images of samples from fluid bed granulation batch no. F22006-002 taken at various spraying intervals during granulation.

FIG. 2 is a series of microscopic images of samples from fluid bed granulation batch no. F22006-003 taken at various spraying intervals during granulation.

FIG. 3 is a histogram showing particle size data collected by sieve analysis of batch nos. F22006-002, F22006-003, and F22006-005.

FIG. 4 is a plot showing the blend uniformity (% label claim) of fluid bed granulation formulation batch no. F22006-002.

FIG. 5 is a plot showing the blend uniformity (% label claim) of fluid bed granulation formulation batch no. F22006-003.

FIG. 6 is a plot showing the blend uniformity (% label claim) of dry blend formulation batch no. F22006-005.

FIG. 7 is a plot showing the dissolution profile of batch no. F22006-002 over time after 1 month of storage at controlled room temperature (CRT) (25° C./60% relative humidity) and accelerated temperature/humidity (ACC) (40° C./75% relative humidity) (only aspirin levels shown).

FIG. 8 is a plot showing the dissolution profile of batch no. F22006-002 over time after 1 month of storage at controlled room temperature (CRT) (25° C./60% relative humidity) and accelerated temperature/humidity (ACC) (40° C./75% relative humidity) (combined aspirin and salicylic acid levels shown).

FIG. 9 is a plot showing the dissolution profile of batch no. F22006-003 over time after 1 month of storage at controlled room temperature (CRT) (25° C./60% relative humidity) compared to initial dissolution profile (only aspirin levels shown).

FIG. 10 is a plot showing the dissolution profile of batch no. F22006-003 over time after 1 month of storage at controlled room temperature (CRT) (25° C./60% relative humidity) compared to initial dissolution profile (combined aspirin and salicylic acid levels shown).

FIG. 11 is a plot showing the dissolution profile of batch no. F22006-005 over time after 1 month of storage at controlled room temperature (CRT) (25° C./60% relative humidity) compared to initial dissolution profile (only aspirin levels shown).

FIG. 12 is a plot showing the dissolution profile of batch no. F22006-005 over time after 1 month of storage at controlled room temperature (CRT) (25° C./60% relative humidity) compared to initial dissolution profile (combined aspirin and salicylic acid levels shown).

FIG. 13 is a flow chart describing an exemplary fluid bed granulation process for manufacturing oral mucosal aspirin formulations.

FIG. 14 is a flow chart describing an exemplary dry blend process for manufacturing oral mucosal aspirin formulations.

### DETAILED DESCRIPTION

As generally described herein, the present disclosure provides, in part, compositions, pharmaceutical formulations, and dosage forms useful for delivering aspirin to a patient in need thereof, e.g., a patient suffering from a disease, disorder, or condition treatable with aspirin, or a healthy patient prophylactically treatable with aspirin. The compositions, pharmaceutical formulations, and dosage forms described herein are formulated for oral mucosal (e.g., sublingual and/or buccal delivery). Also provided herein are methods of treating a subject suffering from a disease, disorder, or condition described herein, the method comprising administering to the subject a formulation described herein. The disclosure also provides methods for making the compositions, pharmaceutical formulations, and dosage forms described herein.

### Definitions

As used herein, “pharmaceutically acceptable excipient,” refers to a substance that aids the administration of an active agent to a subject by, for example, modifying the stability of an active agent or modifying the absorption by a subject upon administration. A pharmaceutically acceptable excipient typically has no significant adverse toxicological effect on the patient. Examples of pharmaceutically acceptable excipients include, for example, water, NaCl (including salt solutions), normal saline solutions, ½ normal saline, sucrose, glucose, bulking agents, buffers, binders, fillers, disintegrants, lubricants, coatings, sweeteners, flavors, alcohols, oils, gelatins, carbohydrates such as amylose or starch, fatty acid esters, hydroxymethycellulose, polyvinyl pyrrolidine, and colors, and the like. One of skill in the art will recognize that other pharmaceutical excipients known in the art are useful in the present invention and include those listed in for example the Handbook of Pharmaceutical Excipients, Rowe R. C., Shesky P. J., and Quinn M. E., 6th Ed., The Pharmaceutical Press, RPS Publishing (2009). The terms “bulking agent,” and “buffer” are used in accordance with the plain and ordinary meaning within the art.

As used herein, “surfactant” refers to a substance that reduces the surface tension of a liquid in which it is dissolved. In some embodiments, a surfactant comprises a water-soluble (hydrophilic) portion and a fat-soluble (lipophilic) portion. In some embodiments, surfactants may be anionic surfactants.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66: 1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “patient” or “subject” to which administration is contemplated includes, but is not limited to, humans (i.e., a male or female of any age group, e.g., a pediatric subject (e.g., adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “patient” and “subject” are used interchangeably herein.

“Disease,” “disorder,” and “condition” are used interchangeably herein.

As used herein, and unless otherwise specified, the terms “treat,” “treating,” and “treatment” contemplate an action that occurs while a subject is suffering from the specified disease, disorder or condition or at risk for the specified disease, disorder, or condition, which reduces the severity of the disease, disorder or condition, or retards or slows the progression and/or emergence of the disease, disorder or condition.

As used herein, the “effective amount” of a compound refers to an amount sufficient to elicit the desired biological or physiologic response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject.

An effective amount encompasses an amount sufficient for therapeutic and/or prophylactic treatment. A therapeutically effective amount and a prophylactically effective amount may differ in certain embodiments.

As used herein, the term “particle size” is defined as the diameter of a particle as determined by a particle size analyzer.

As used herein, the term “Cₘₐₓ” refers to the maximum plasma concentration of a compound described herein (e.g., aspirin) after administration of a formulation containing the compound (e.g., a formulation disclosed herein) to a subject. The term “Cₘₐₓ/D” refers to the Cₘₐₓ divided by the dose of the compound contained in the administered formulation.

As used herein, the term “Tₘₐₓ” refers to time to Cₘₐₓ following administration of a formulation described herein to a subject.

As used herein, the term “AUC” refers to the area under the plasma concentration vs. time curve from zero to infinity for a compound described herein (e.g., the area under the aspirin plasma concentration vs. time curve) after administration of a formulation containing the compound (e.g., a formulation disclosed herein) to a subject. The term “AUC/D” refers to the AUC divided by the dose of the compound contained in the administered formulation.

### Compounds

Described herein are pharmaceutical formulations or dosage forms comprising aspirin, represented by formula (I):

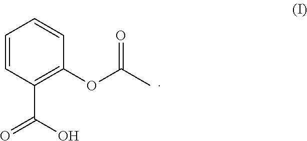

The compound of formula (I) may be referred to as aspirin, 2-acetoxybenzoic acid, acetylsalicylate, acetylsalicylic acid, and ASA, and these terms are used interchangeable herein. Aspirin may exist in a crystalline solid form comprising white crystals, commonly tabular or needle-like, or white crystalline powder. Aspirin has a melting point of 135° C. (PubChem).

Aspirin contains a carboxylic acid group and has a pKa of approximately 3.5 (PubChem). Aspirin has a log p value of 1.19 (PubChem).

Aspirin is classified as a Biopharmaceutics Class System (BCS) Class I drug. By definition, BCS Class I drugs are highly absorbed, and have neither solubility nor permeability-limited absorption (United States Food and Drug Administration).

### Pharmaceutical Formulations and Dosage Forms

In one aspect, the present disclosure provides pharmaceutical formulations or dosage forms useful for preventing and/or treating a disease, disorder, or condition described herein. The disclosure provides pharmaceutical formulations for oral mucosal (e.g., sublingual and/or buccal) delivery that contain aspirin as the active ingredient, and one or more pharmaceutically acceptable excipients, including a buffer and a surfactant.

Oral mucosal (e.g., sublingual and/or buccal) delivery utilizes the many capillaries found in the cheek and under the tongue (e.g., within the oral cavity) to quickly introduce a drug into the circulatory system of a patient. Oral mucosal (e.g., sublingual and/or buccal) delivery has the added benefit of precluding direct physical/chemical contact with digestive tract mucosa and also avoiding first pass metabolism by the digestive tract and the liver. Oral mucosal delivery is a preferred delivery method for drugs that do not absorb efficiently in the stomach and/or digestive tract, including the rectum, or that cannot be given intravenously, subcutaneously, intramuscularly and or intrathecally.

Oral mucosal (e.g., sublingual and/or buccal) delivery of aspirin allows for rapid introduction of the drug into the circulatory system of a patient in need thereof. This approach represents a significant improvement in patient outcomes due to aspirin's antithrombotic, antipyretic, and anti-inflammatory properties, and associated use in treating conditions in which a patient would benefit from rapid and effective delivery of aspirin. An exemplary patient that would benefit from rapid delivery of aspirin is a geriatric orthopedic surgery patient who fails a swallow test and prefers not to be injected with low molecular weight heparin and is in need of venous thrombosis prophylaxis.

Aspirin contains a carboxylic acid functional group and has a pKa of approximately 3.5. In aqueous solution, aspirin is in equilibrium with its conjugate base. This acid-conjugate base equilibrium is represented by Equation 1 below.

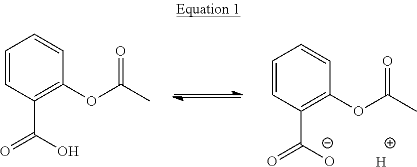

In its conjugate base/ionized form, aspirin is unable to efficiently cross the mucous membranes in the oral cavity to enter the blood stream, rendering oral mucosal (e.g., sublingual and/or buccal) delivery of ionized aspirin inefficient. The pH of saliva in the oral cavity ranges between 6.2 to 7.6 (Dawood, I. M.; El-Samarrai, S. K. Saliva and Oral Health. Int. J. Adv. Res. Biol. Sci. 2018, 5, 1-45) depending on the individual, with an average pH of 6.7. The Henderson-Hasselbalch equation, represented by Equation 2 below, provides the ratio of conjugate base to acid at a given pH of an acid having a given pKa.

$$pH = p {K}_{a} + {log}_{10} ( \frac{[ Base ]}{[ Acid ]} ) \qquad Equation 2$$

Thus, in an aqueous environment having a pH of 6.7 (i.e., commensurate to the pH of the saliva of an average human subject), aspirin, having a pKa of 3.5, would exist in its ionized/conjugate base form at a ratio of 1584 molecules of ionized conjugate base relative to 1 molecule of non-ionized acid and would be unable to efficiently enter a patient's circulatory system through oral mucosal (e.g., sublingual and/or buccal) routes. Exemplary calculations are provided below.

Using the lower saliva pH limit of 6.2 in equation 1:

$$6.2 = 3.5 + {log}_{10} [ {A}^{-} / HA ]$$

$$Thus , {log}_{10} [ {A}^{-} / HA ] = 2.7$$

$$Therefore , [ {A}^{-} / HA ] = 1 {0}^{2.7} = 501$$

For every one molecule of non-ionized aspirin that can enter the blood stream, there are 501 ionized aspirin molecules that cannot cross the mucosal membranes to enter the sublingual capillaries.

Using the upper saliva pH limit of 7.6 in equation 1:

$$7.6 = 3.5 + {log}_{10} [ {A}^{-} / HA ]$$

$$Thus , {log}_{10} ⌊ {A}^{-} / HA ⌋ = 4.1$$

$$Therefore , [ {A}^{-} / HA ] = 1 {0}^{4.1} = 12589$$

For every one molecule of non-ionized aspirin that can enter the blood stream, there are 12589 ionized aspirin molecules that cannot cross the mucosal membranes to enter the sublingual capillaries.

Using the average saliva pH value of 6.7 in equation 1:

$$6. 7 = 3.5 + {log}_{10} [ {A}^{-} / HA ]$$

$$Thus , {log}_{10} [ {A}^{-} / HA ] = 3.2$$

$$Therefore , [ {A}^{-} / HA ] = 1 {0}^{3.2} = 1584$$

For every one molecule of non-ionized aspirin that can enter the blood stream, there are 1584 ionized aspirin molecules that cannot cross the mucosal membranes to enter the sublingual capillaries.

The inventors have developed pharmaceutical formulations for the oral mucosal (e.g., sublingual and/or buccal) delivery of aspirin that overcome problems associated with the relatively high pH of the oral cavity and resulting ionization of aspirin. The pharmaceutical formulations of the present disclosure comprise aspirin, a buffer, a surfactant, and one or more pharmaceutically acceptable excipients. When administered to a patient through the oral mucosa (e.g., sublingually and/or buccally), the buffer component reduces the local pH within the oral cavity and maintains a reduced pH throughout the duration of drug delivery. The reduced pH causes the aspirin to exist primarily in its non-ionized form throughout administration, thus enabling delivery across the mucous membranes of the oral cavity and directly into the circulatory system of the patient. The surfactant reduces salivary protein binding of the aspirin, thereby further increasing absorption efficiency. The pharmaceutical formulations provided herein are also optimized for rapid dissolution (e.g., by utilizing micronized aspirin in the formulation), which further enhances the efficiency of oral mucosal (e.g., sublingual and/or buccal) delivery.

In one aspect, provided herein is a pharmaceutical formulation for oral mucosal (e.g., sublingual and/or buccal) administration comprising:
70 mg to 90 mg micronized aspirin;
a buffer;
a surfactant;
and one or more pharmaceutically acceptable excipients,
wherein the pH of a solution of the formulation dissolved in 1 mL distilled water is less than 3.

In another aspect, provided herein is a pharmaceutical formulation for oral mucosal (e.g., sublingual and/or buccal) administration comprising:
70 mg to 90 mg aspirin;
a buffer;
a surfactant;
and one or more pharmaceutically acceptable excipients,
wherein the pH of a solution of the formulation dissolved in 1 mL distilled water is less than 3.

In some embodiments, the formulation comprises granules comprising an intragranular fraction, wherein the intragranular fraction comprises the aspirin. In some embodiments, the intragranular fraction further comprises the buffer. In some embodiments, the intragranular fraction further comprises one or more pharmaceutically acceptable excipients (e.g., aspartame).

In some embodiments, at least 70% of the granules are 105 microns in diameter or smaller. In some embodiments, at least 75% of the granules are 105 microns in diameter or smaller. In some embodiments, at least 80% of the granules are 105 microns in diameter or smaller. In some embodiments, at least 85% of the granules are 105 microns in diameter or smaller. In some embodiments, at least 90% of the granules are 105 microns in diameter or smaller. Granule size may be determined by methods known in the art, e.g., by using a sieve shaker or a particle size analyzer after suspending the granules in a liquid phase in which they are insoluble or sparingly soluble (such as, e.g., an alkane such as, e.g., hexane).

In some embodiments, the formulation does not comprise granules.

In some embodiments, the amount of aspirin in the formulation is 73 mg to 89 mg, 74 mg to 88 mg, 75 mg to 87 mg, 76 mg to 86 mg, 77 mg to 85 mg, 78 mg to 84 mg, 79 mg to 83 mg, or 80 mg to 82 mg. In some embodiments, the amount of aspirin in the formulation is 80 mg to 90 mg, 80 mg to 88 mg, 80 mg to 86 mg, 80 mg to 84 mg, or 80 mg to 82 mg.

In some embodiments, the amount of aspirin in the formulation is 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg. In certain embodiments, the amount of aspirin in the formulation is 80 mg to 82 mg.

In some embodiments, the amount of aspirin in the formulation is 75 mg to 85 mg. In certain embodiments, the amount of aspirin in the formulation is 81 mg.

In some embodiments, the micronized aspirin in the formulation has a median particle size of 1 μm to 20 μm, 1 μm to 18 μm, 1 μm to 16 μm, 1 μm to 14 μm, 1 μm to 12 μm, 1 μm to 10 μm, 1 μm to 8 μm, 1 μm to 6 μm, or 1 μm to 4 μm. In some embodiments, the micronized aspirin in the formulation has a median particle size of 1 μm to 10 μm. In some embodiments, the micronized aspirin in the formulation has a median particle size of less than 10 μm.

Determination of particle size may be accomplished by methods known in the art, e.g., by using a particle size analyzer and suspending the particles in a liquid phase in which they are insoluble or sparingly soluble, such as, e.g., an alkane such as, e.g., hexane

In some embodiments, the pH of a solution of the formulation dissolved in 1 mL distilled water is 2 to 3, 2 to 2.9, 2 to 2.8, 2 to 2.7, 2 to 2.6, 2 to 2.5, 2 to 2.4, 2 to 2.3, or 2 to 2.2. In some embodiments, the pH of a solution of the formulation dissolved in 1 mL distilled water is 2 to 3, 2.1 to 3, 2.2 to 3, 2.3 to 3, 2.4 to 3, 2.5 to 3, 2.6 to 3, 2.7 to 3, 2.8 to 3, or 2.9 to 3.

In some embodiments, the pH of a solution of the formulation dissolved in 1 mL saliva is 2 to 3, 2 to 2.9, 2 to 2.8, 2 to 2.7, 2 to 2.6, 2 to 2.5, 2 to 2.4, 2 to 2.3, or 2 to 2.2. In some embodiments, the pH of a solution of the formulation dissolved in 1 mL saliva is 2 to 3, 2.1 to 3, 2.2 to 3, 2.3 to 3, 2.4 to 3, 2.5 to 3, 2.6 to 3, 2.7 to 3, 2.8 to 3, or 2.9 to 3.

In some embodiments, the pH of a solution of the formulation dissolved in 1 mL distilled water is 2, 2.1, 2.2, 2.3, 2.4, 2.5, 2.6, 2.7, 2.8, 2.9, or 3.

In some embodiments, the pH of a solution of the formulation dissolved in 1 mL saliva is 2, 2.1, 2.2, 2.3, 2.4, 2.5, 2.6, 2.7, 2.8, 2.9, or 3.

In some embodiments, the pH of a solution of the formulation dissolved in 1 mL distilled water is less than 2.9. In some embodiments, the pH of a solution of the formulation dissolved in 1 mL distilled water is less than 2.8. In some embodiments, the pH of a solution of the formulation dissolved in 1 mL distilled water is less than 2.7. In some embodiments, the pH of a solution of the formulation dissolved in 1 ml distilled water is less than 2.6. In some embodiments, the pH of a solution of the formulation dissolved in 1 mL distilled water is less than 2.5. In some embodiments, the pH of a solution of the formulation dissolved in 1 mL distilled water is less than 2.4. In some embodiments, the pH of a solution of the formulation dissolved in 1 mL distilled water is less than 2.3. In some embodiments, the pH of a solution of the formulation dissolved in 1 mL distilled water is less than 2.2. In some embodiments, the pH of a solution of the formulation dissolved in 1 mL distilled water is less than 2.1. In some embodiments, the pH of a solution of the formulation dissolved in 1 mL distilled water is 2 to 2.5. In some embodiments, the pH of a solution of the formulation dissolved in 1 ml distilled water is 1.8 to 2.5.

In some embodiments, the pH of a solution of the formulation dissolved in 1 mL saliva is less than 2.9. In some embodiments, the pH of a solution of the formulation dissolved in 1 mL saliva is less than 2.8. In some embodiments, the pH of a solution of the formulation dissolved in 1 mL saliva is less than 2.7. In some embodiments, the pH of a solution of the formulation dissolved in 1 mL saliva is less than 2.6. In some embodiments, the pH of a solution of the formulation dissolved in 1 mL saliva is less than 2.5. In some embodiments, the pH of a solution of the formulation dissolved in 1 mL saliva is less than 2.4. In some embodiments, the pH of a solution of the formulation dissolved in 1 mL saliva is less than 2.3. In some embodiments, the pH of a solution of the formulation dissolved in 1 mL saliva is less than 2.2. In some embodiments, the pH of a solution of the formulation dissolved in 1 mL saliva is less than 2.1. In some embodiments, the pH of a solution of the formulation dissolved in 1 mL saliva is 2 to 2.5. In some embodiments, the pH of a solution of the formulation dissolved in 1 mL saliva is 1.8 to 2.5.

Determination of the pH of a solution of a formulation disclosed herein may be accomplished by methods known in the art, e.g., as described herein and by standard protocols, e.g., by protocols described in the United States Pharmacopoeia, Chapter <791>.

In some embodiments, the buffer concentration of the formulation is at least 50 mM, at least 75 mM, at least 100 mM, at least 125 mM, at least 150 mM, at least 200 mM, at least 225 mM, at least 250 mM, at least 300 mM, at least 325 mM, at least 350 mM, at least 375 mM, at least 400 mM, at least 425 mM, at least 450 mM, at least 475 mM, or at least 500 mM when dissolved in 1 mL of distilled and/or deionized water.

In some embodiments, the buffer concentration of the formulation is 100 mM to 500 mM, 150 mM to 500 mM, 200 mM to 500 mM, 250 mM to 500 mM, 300 mM to 500 mM, 350 mM to 500 mM, 400 mM to 500 mM, or 450 mM to 500 mM when dissolved in 1 mL of distilled and/or deionized water. In some embodiments, the buffer concentration of the formulation is 100 mM to 500 mM, 100 mM to 450 mM, 100 mM to 400 mM, 100 mM to 350 mM, 100 mM to 300 mM, 100 mM to 250 mM, 100 mM to 200 mM, or 100 mM to 150 mM when dissolved in 1 mL of distilled and/or deionized water. In some embodiments, the buffer concentration of the formulation is 100 mM to 500 mM, 150 mM to 450 mM, 200 mM to 400 mM, or 250 mM to 350 mM when dissolved in 1 mL of distilled and/or deionized water.

In some embodiments, the buffer concentration of the formulation is 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, or 500 mM when dissolved in 1 mL of distilled and/or deionized water.

In some embodiments, the buffer concentration of the formulation is at least 100 mM when dissolved in 1 mL of distilled and/or deionized water. In some embodiments, the buffer concentration of the formulation is at least 150 mM when dissolved in 1 mL of distilled and/or deionized water. In some embodiments, the buffer concentration of the formulation is at least 200 mM when dissolved in 1 mL of distilled and/or deionized water. In some embodiments, the buffer concentration of the formulation is at least 250 mM when dissolved in 1 mL of distilled and/or deionized water. In some embodiments, the buffer concentration of the formulation is at least 300 mM when dissolved in 1 mL of distilled water. In some embodiments, the buffer concentration of the formulation is at least 350 mM when dissolved in 1 mL of distilled and/or deionized water. In some embodiments, the buffer concentration of the formulation is at least 400 mM when dissolved in 1 mL of distilled and/or deionized water. In some embodiments, the buffer concentration of the formulation is at least 450 mM when dissolved in 1 mL of distilled and/or deionized water. In some embodiments, the buffer concentration of the formulation is at least 500 mM when dissolved in 1 mL of distilled and/or deionized water.

As used herein, dissolution of a formulation of the disclosure encompasses both full and partial dissolution of the formulation in a solvent (e.g., distilled water). Solutions of the formulations disclosed herein encompass both mixtures in which the formulation has fully dissolved and mixtures in which the formulation has partially dissolved. In some embodiments, the formulation is at least 50%, at least 55%, at least 60%, at least 65%, at least 70%, at least 75%, at least 80%, at least 85%, at least 90%, or at least 95% dissolved in the solutions described herein. In some embodiments, the formulation is at least 90% dissolved in the solutions described herein. In some embodiments, the formulation is at least 95% dissolved in the solutions described herein. Percent dissolution is the fraction of the dose that has gone into solution within a specified time period as measured by a dissolution test as described herein.

In some embodiments, the buffer comprises citric acid, or a hydrate thereof, and a citrate salt, or a hydrate thereof. In some embodiments, the citric acid or hydrate thereof is selected from citric acid monohydrate and citric acid anhydrous. In certain embodiments, the citric acid or hydrate thereof is citric acid anhydrous. In certain embodiments, the citric acid or hydrate thereof is citric acid monohydrate. In some embodiments, the citrate salt or hydrate thereof is selected from the group consisting of monosodium citrate, disodium citrate, trisodium citrate, monopotassium citrate, dipotassium citrate, and tri-potassium citrate, or a hydrate thereof. In certain embodiments, the citrate salt or hydrate thereof is tri-potassium citrate monohydrate. In certain embodiments, the citrate salt or hydrate thereof is mono-potassium citrate or a hydrate thereof.

In some embodiments, the buffer consists of citric acid, or a hydrate thereof, and a citrate salt, or a hydrate thereof. In some embodiments, the citric acid or hydrate thereof is selected from citric acid monohydrate and citric acid anhydrous. In certain embodiments, the citric acid or hydrate thereof is citric acid anhydrous. In certain embodiments, the citric acid or hydrate thereof is citric acid monohydrate. In some embodiments, the citrate salt or hydrate thereof is selected from the group consisting of monosodium citrate, disodium citrate, trisodium citrate, monopotassium citrate, dipotassium citrate, and tri-potassium citrate, or a hydrate thereof. In certain embodiments, the citrate salt or hydrate thereof is tri-potassium citrate monohydrate. In certain embodiments, the citrate salt or hydrate thereof is mono-potassium citrate or a hydrate thereof.

In some embodiments, the buffer is 20% to 50%, 25% to 50%, 35% to 50%, 20% to 45%, 20% to 40%, 20% to 35%, 25% to 45%, or 30% to 40% of the formulation by mass. In certain embodiments, the buffer is 30% to 40% of the formulation by mass. In some embodiments, the buffer is 30%, 32%, 34%, 36%, 38%, or 40% of the formulation by mass. In certain embodiments, the buffer is 36% of the formulation by mass.

In some embodiments, the surfactant is an anionic surfactant. In some embodiments, the surfactant is selected from the group consisting of sodium lauryl sulfate, ammonium lauryl sulfate, potassium lauryl sulfonate, potassium lauryl phosphate, sodium deoxycholate, and potassium deoxycholate. In certain embodiments, the surfactant is sodium lauryl sulfate.

In some embodiments, the anionic surfactant (e.g., sodium lauryl sulfate) increases the percentage of oral mucosal (e.g., sublingual and/or buccal) absorption of aspirin in a subject administered the formulation oral mucosally (e.g., sublingually and/or buccally) compared to a subject administered an oral mucosal aspirin formulation that does not comprise an anionic surfactant.

In some embodiments, the anionic surfactant increases the percentage of oral mucosal (e.g., sublingual and/or buccal) absorption of aspirin in a subject administered the formulation oral mucosally (e.g., sublingually and/or buccally) compared to a subject administered an oral mucosal aspirin formulation that does not comprise an anionic surfactant by 1% to 5%, 5% to 10%, 10% to 15%, or 15% to 20%. In some embodiments, the anionic surfactant increases by 10% to 20% the percentage of oral mucosal (e.g., sublingual and/or buccal) absorption of aspirin in a subject administered the formulation oral mucosally (e.g., sublingually and/or buccally) compared to a subject administered an oral mucosal aspirin formulation that does not comprise an anionic surfactant. In some embodiments, the anionic surfactant increases by at least 10% or at least 20% the percentage of oral mucosal (e.g., sublingual and/or buccal) absorption of aspirin in a subject administered the formulation oral mucosally (e.g., sublingually and/or buccally) compared to a subject administered an oral mucosal aspirin formulation that does not comprise an anionic surfactant.

In some embodiments, the surfactant is 1% to 7%, 1.5% to 7%, 2% to 7%, 2.5% to 7%, 3% to 7%, 1% to 6.5%, 1% to 6%, 1% to 5.5%, 1% to 5%, 1% to 4.5%, 1% to 4%, 1.5% to 6.5%, 2% to 6%, 2.5% to 5.5%, or 3% to 5% of the formulation by mass. In certain embodiments, the surfactant is 3% to 4% of the formulation by mass. In some embodiments, the surfactant is 2%, 2.2%, 2.4%, 2.6%, 2.8%, 3%, 3.2%, 3.4%, 3.6%, 3.8%, 4%, 4.2%, 4.4%, or 4.6% of the formulation by mass. In certain embodiments, the surfactant is 3.6% of the formulation by mass.

In another aspect, provided herein is a pharmaceutical formulation for oral mucosal (e.g., sublingual and/or buccal) administration comprising:
70 mg to 90 mg micronized aspirin;
56 mg to 75 mg citric acid anhydrous;
1 mg to 5 mg tri-potassium citrate monohydrate;
1 mg to 10 mg sodium lauryl sulfate; and
one or more pharmaceutically acceptable excipients.

In another aspect, provided herein is a pharmaceutical formulation for oral mucosal (e.g., sublingual and/or buccal) administration comprising:
70 mg to 90 mg aspirin;
56 mg to 75 mg citric acid anhydrous;
1 mg to 5 mg tri-potassium citrate monohydrate;
1 mg to 10 mg sodium lauryl sulfate; and
one or more pharmaceutically acceptable excipients.

In some embodiments, the formulation comprises granules comprising an intragranular fraction, wherein the intragranular fraction comprises the aspirin. In some embodiments, the intragranular fraction further comprises the citric acid anhydrous and the tri-potassium citrate monohydrate. In some embodiments, the intragranular fraction further comprises one or more pharmaceutically acceptable excipients (e.g., aspartame).

In some embodiments, the formulation does not comprise granules.

In another aspect, provided herein is a pharmaceutical formulation for oral mucosal (e.g., sublingual and/or buccal) administration comprising:
81 mg micronized aspirin;
64 mg citric acid anhydrous;
2.4 mg tri-potassium citrate monohydrate;
6.8 mg sodium lauryl sulfate; and
one or more pharmaceutically acceptable excipients.

In another aspect, provided herein is a pharmaceutical formulation for oral mucosal (e.g., sublingual and/or buccal) administration comprising:
81 mg aspirin;
64 mg citric acid anhydrous;
2.4 mg tri-potassium citrate monohydrate;
6.8 mg sodium lauryl sulfate; and
one or more pharmaceutically acceptable excipients.

In some embodiments, the formulation comprises granules comprising an intragranular fraction, wherein the intragranular fraction comprises the aspirin. In some embodiments, the intragranular fraction further comprises the citric acid anhydrous and the tri-potassium citrate monohydrate. In some embodiments, the intragranular fraction further comprises one or more pharmaceutically acceptable excipients (e.g., aspartame).

In some embodiments, at least 70% of the granules are 105 microns in diameter or smaller. In some embodiments, at least 75% of the granules are 105 microns in diameter or smaller. In some embodiments, at least 80% of the granules are 105 microns in diameter or smaller. In some embodiments, at least 85% of the granules are 105 microns in diameter or smaller. In some embodiments, at least 90% of the granules are 105 microns in diameter or smaller. Granule size may be determined by methods known in the art, e.g., by using a sieve shaker or a particle size analyzer after suspending the granules in a liquid phase in which they are insoluble or sparingly soluble (such as, e.g., an alkane such as, e.g., hexane).

In some embodiments, the formulation does not comprise granules.

In some embodiments, at least 50% of the aspirin is released (i.e., dissolved) within 2 minutes using a paddle dissolution apparatus (e.g., a USP <711> Type 2 dissolution apparatus). In some embodiments, at least 55% of the aspirin is released within 2 minutes using a paddle dissolution apparatus (e.g., a USP <711> Type 2 dissolution apparatus). In some embodiments, at least 60% of the aspirin is released within 2 minutes using a paddle dissolution apparatus (e.g., a USP <711> Type 2 dissolution apparatus). In some embodiments, at least 65% of the aspirin is released within 2 minutes using a paddle dissolution apparatus (e.g., a USP <711> Type 2 dissolution apparatus). In some embodiments, at least 70% of the aspirin is released within 2 minutes using a paddle dissolution apparatus (e.g., a USP <711> Type 2 dissolution apparatus). In some embodiments, at least 75% of the aspirin is released within 2 minutes using a paddle dissolution apparatus (e.g., a USP <711> Type 2 dissolution apparatus). In some embodiments, at least 80% of the aspirin is released within 2 minutes using a paddle dissolution apparatus (e.g., a USP <711> Type 2 dissolution apparatus). In some embodiments, at least 85% of the aspirin is released within 2 minutes using a paddle dissolution apparatus (e.g., a USP <711> Type 2 dissolution apparatus). In some embodiments, at least 50% of the aspirin is released within 5 minutes using a paddle dissolution apparatus (e.g., a USP <711> Type 2 dissolution apparatus). In some embodiments, at least 55% of the aspirin is released within 5 minutes using a paddle dissolution apparatus (e.g., a USP <711> Type 2 dissolution apparatus). In some embodiments, at least 60% of the aspirin is released within 5 minutes using a paddle dissolution apparatus (e.g., a USP <711> Type 2 dissolution apparatus). In some embodiments, at least 65% of the aspirin is released within 5 minutes using a paddle dissolution apparatus (e.g., a USP <711> Type 2 dissolution apparatus). In some embodiments, at least 70% of the aspirin is released within 5 minutes using a paddle dissolution apparatus (e.g., a USP <711> Type 2 dissolution apparatus). In some embodiments, at least 75% of the aspirin is released within 5 minutes using a paddle dissolution apparatus (e.g., a USP <711> Type 2 dissolution apparatus). In some embodiments, at least 80% of the aspirin is released within 5 minutes using a paddle dissolution apparatus (e.g., a USP <711> Type 2 dissolution apparatus). In some embodiments, at least 85% of the aspirin is released within 5 minutes using a paddle dissolution apparatus (e.g., a USP <711> Type 2 dissolution apparatus).

In some embodiments, at least 70% of the aspirin is released within 10 minutes using a paddle dissolution apparatus (e.g., a USP <711> Type 2 dissolution apparatus). In some embodiments, at least 75% of the aspirin is released within 10 minutes using a paddle dissolution apparatus (e.g., a USP <711> Type 2 dissolution apparatus). In some embodiments, at least 80% of the aspirin is released within 10 minutes using a paddle dissolution apparatus (e.g., a USP <711> Type 2 dissolution apparatus). In some embodiments, at least 85% of the aspirin is released within 10 minutes using a paddle dissolution apparatus (e.g., a USP <711> Type 2 dissolution apparatus).

In some embodiments, at least 80% of the aspirin is released within 30 minutes using a paddle dissolution apparatus (e.g., a USP <711> Type 2 dissolution apparatus). In some embodiments, at least 85% of the aspirin is released within 30 minutes using a paddle dissolution apparatus (e.g., a USP <711> Type 2 dissolution apparatus). In some embodiments, at least 90% of the aspirin is released within 30 minutes using a paddle dissolution apparatus (e.g., a USP <711> Type 2 dissolution apparatus). In some embodiments, at least 95% of the aspirin is released within 30 minutes using a paddle dissolution apparatus (e.g., a USP <711> Type 2 dissolution apparatus).

Determination of the dissolution rate of a formulation disclosed herein may be accomplished by methods known in the art and as described herein, e.g., using a USP <711> Type 2 dissolution apparatus.

In some embodiments, the formulation comprises an anti-settling and/or anti-sagging agent. In certain embodiments, the anti-settling and/or anti-sagging agent is fumed silica.

In some embodiments, the anti-settling and/or anti-sagging agent is 5% to 13%, 5% to 12%, 5% to 11%, 5% to 10%, 5% to 9%, 6% to 13%, 7% to 13%, 8% to 13%, 6% to 12%, 7% to 11%, or 8% to 10% of the formulation by mass. In certain embodiments, the anti-settling and/or anti-sagging agent is 8% to 10% of the formulation by mass. In some embodiments, the anti-settling and/or anti-sagging agent is 5%, 6%, 7%, 8%, 9%, 10%, 11%, 12%, or 13% of the formulation by mass. In certain embodiments, the anti-settling and/or anti-sagging agent is 9% of the formulation by mass.

In some embodiments, the formulation comprises one or more of: aspartame, sodium lauryl sulfate, and fumed silica. In some embodiments, the formulation comprises each of aspartame, sodium lauryl sulfate, and fumed silica.

In some embodiments, the formulation is a solid dosage form. In some embodiments, the formulation is in powder form. In some embodiments, the formulation is in tablet form.

In another aspect, provided herein is a pharmaceutical formulation for oral mucosal (e.g., sublingual and/or buccal) administration comprising:
70 mg to 90 mg micronized aspirin;
a buffer;
a surfactant;
and one or more pharmaceutically acceptable excipients,
wherein the pH of a solution of the formulation dissolved in 1 mL distilled water is less than 3,
wherein the formulation is manufactured using a process comprising:
   mixing the aspirin with one or more pharmaceutically acceptable excipients to form a mixture;
   micronizing the mixture to form a micronized aspirin pre-blend;
   wet granulating the micronized aspirin pre-blend with a granulation fluid to form a granulated composition; and
   blending the granulated composition with one or more pharmaceutically acceptable excipients to afford the formulation.

In some embodiments, the aspirin is mixed with at least citric acid anhydrous, tri-potassium citrate monohydrate, and a surfactant. In certain embodiments, the surfactant is an anionic surfactant. In certain embodiments, the surfactant is sodium lauryl sulfate. In some embodiments, mixing the aspirin with the pharmaceutically acceptable excipients prior to micronization achieves improved uniform distribution of aspirin in the blend.

In some embodiments, the granulation fluid is aqueous. In some embodiments, the granulation fluid comprises a surfactant. In certain embodiments, the surfactant is an anionic surfactant. In certain embodiments, the surfactant is sodium lauryl sulfate.

In some embodiments, the granulated composition is blended with an anti-settling and/or anti-sagging agent. In some embodiments, the anti-settling agent is fumed silica.

In another aspect, provided herein is a pharmaceutical formulation for oral mucosal (e.g., sublingual and/or buccal) administration comprising:
70 mg to 90 mg micronized aspirin;
a buffer;
a surfactant;
and one or more pharmaceutically acceptable excipients,
wherein the pH of a solution of the formulation dissolved in 1 mL distilled water is less than 3,
wherein the formulation is manufactured using a process comprising:
   mixing the aspirin with one or more pharmaceutically acceptable excipients to form a mixture;
   micronizing the mixture to form a micronized aspirin dry blend; and
   blending the micronized aspirin dry blend with one or more pharmaceutically acceptable excipients to afford the formulation.

In some embodiments, the aspirin is mixed with at least citric acid anhydrous, tri-potassium citrate monohydrate, and a surfactant. In certain embodiments, the surfactant is an anionic surfactant. In certain embodiments, the surfactant is sodium lauryl sulfate. In some embodiments, mixing the aspirin with the pharmaceutically acceptable excipients prior to micronization achieves improved uniform distribution of aspirin in the blend.

In some embodiments, the micronized aspirin dry blend is blended with an anti-settling and/or anti-sagging agent. In some embodiments, the anti-settling and/or anti-sagging agent is fumed silica.

### Routes of Administration

The pharmaceutical formulations described herein can be administered via oral mucosal delivery. In an embodiment, the pharmaceutical formulations described herein are administered to a subject in need thereof in the form of a powder. In an embodiment, the pharmaceutical formulations described herein are administered to a subject in need thereof in the form of a tablet. In some embodiments, the pharmaceutical formulations described herein are administered supralingually. In some embodiments, the pharmaceutical formulations described herein are administered sublingually. In some embodiments, the pharmaceutical formulations described herein are administered buccally.

### Methods of Treatment

In one aspect, provided herein are methods of treating a disease, disorder, or condition, or reducing the symptoms of said disease, disorder, or condition, comprising administering to a patient in need thereof a pharmaceutical formulation described herein. In some embodiments, the disease, disorder, or condition is selected from pain, fever, and swelling. In some embodiments, the disease, disorder, or condition is a bacterial or viral infection, and the method reduces the symptoms of the bacterial or viral infection. In some embodiments, the method provides an anti-platelet effect. In some embodiments, the method provides an anti-inflammatory effect. In some embodiments, the method provides an anti-pyretic effect. In some embodiments, the method provides an anti-thrombotic effect.

In some embodiments, the methods provided herein comprise oral-mucosally (e.g., sublingually and/or buccally) administering a pharmaceutical formulation described herein to a subject in need thereof.

Contemplated methods may include administering a pharmaceutical formulation described herein once per week, twice per week, three times per week, four times per week, five times per week, six times per week, once a day, twice a day, three times a day, four times a day, or five times a day. In some embodiments, a pharmaceutical formulation described herein may be administered once per day. In some embodiments, a formulation may be administered for 1 minute, 2 minutes, 3 minutes, 4 minutes, 5 minutes, 6 minutes, 7 minutes, 8 minutes, 9 minutes, or 10 minutes.

### Kits

In one aspect, the disclosure provides a kit for the treatment of a disease, disorder, or condition, or reducing the symptoms of said disease, disorder, or condition, described herein, comprising a pharmaceutical formulation described herein.

In one aspect, provided herein is a kit for suitable storage of a pharmaceutical formulation described herein. In some embodiments, the kit comprises a pouch suitable for packaging any of the pharmaceutical formulations provided herein.

Provided herein, in an embodiment, is a product including a pharmaceutical formulation described herein contained in a kit. Contemplated kits may include a container. In some embodiments, the kit comprises a primary container and a secondary container. In some embodiments, the primary container is a pouch. In some embodiments, the pouch comprises a single dose of a pharmaceutical formulation disclosed herein. In some embodiments, the single dose of a pharmaceutical formulation is a powder for oral mucosal (e.g., sublingual and/or buccal delivery). In some embodiments, the secondary container is a box. In some embodiments, a box contains 1, 2, 3, 4, 5, 6, 7, 8, 9, or 10 pouched powder dosage forms for oral mucosal (e.g., sublingual and/or buccal) delivery. In some aspects, contemplated kits may comprise instructions for use in treating a disease, disorder, or condition described herein and/or provide instructions for storage, e.g., instructions to store between 15° C. to 30° C.

### EXEMPLIFICATION

The representative examples that follow are intended to help illustrate the disclosure, and are not intended to, nor should they be construed to, limit the scope of the disclosure.

### Abbreviations

API active pharmaceutical ingredient
BU blend uniformity
CU content uniformity
FB/FBG fluid bed/fluid bed granulation
HPLC high performance liquid chromatography
RPM revolutions per minute
SLS sodium lauryl sulfate
T₀ beginning time
T_{end} end time
USP United States Pharmacopoeia

### Example 1. Granulation, Blending, and Blend Uniformity of Sublingual Aspirin Formulations

The example describes micronization and granulation processes used to manufacture aspirin formulations for oral mucosal (e.g., sublingual and/or buccal) delivery, as well as blend uniformity analysis. The composition of the formulations for oral-mucosal (e.g., sublingual or buccal) delivery is provided in Table 1.

TABLE 1
Formulation Composition (100 g)

| | | | | Amount |
|---|---|---|---|---|
| | | Amount | | per dose |
| Ingredients | Function | (g) | % w/w | (mg) |
| Aspirin USP 80 mesh | API | 43.41 | 43.41 | 81.00 |
| Powder | | | | |
| Citric Acid | pH adjustment | 34.27 | 34.27 | 63.95 |
| anhydrous, powder | conjugate acid | | | |
| | (buffer reagent) | | | |
| Tri-potassium citrate | conjugate base | 1.31 | 1.31 | 2.44 |
| monohydrate | (buffer reagent) | | | |
| Aspartame 100 mesh | taste masking | 8.68 | 8.68 | 16.20 |
| powder | agent | | | |
| Sodium Lauryl Sulfate | Surfactant or | 3.65 | 3.65 | 6.81 |
| | detergent/wetting | | | |
| | agent | | | |
| Aerosil 200 Pharma | anti-settling/ | 8.68 | 8.68 | 16.20 |
| | anti-sagging agent | | | |
| Total | | 100.00 | 100.00 | 186.60 |

API and excipients were obtained from reputable commercial sources and were USP quality controlled. Aspirin USP 80 mesh powder was obtained from Jilin Pharmaceutical USA Inc. (Manalapan, NJ 07726). Citric acid anhydrous powder was obtained from Primary Products Ingredients Americas LLC (Vista, CA).

An oral mucosal (e.g., sublingual and/or buccal) aspirin formulation was prepared using a process comprising fluid bed granulation. Aspirin USP 80 mesh powder, tri-potassium citrate monohydrate, and aspartame 100 mesh powder were sieved using a Porta Sifter screener. The resulting mixture was blended with citric acid anhydrous powder and sodium lauryl sulfate using a V blender, and the blended mixture was micronized using a jet mill micronizer (Sturtevant Micronizer) to afford a micronized aspirin pre-blend. The micronized aspirin pre-blend was granulated using top spray granulation in a Glatt GPCG 3.1 fluid bed dryer, using a granulation fluid containing purified water USP and 0.3% sodium lauryl sulfate to afford a granulated composition. The granulated composition was then sieved with Aerosil 200 Pharma using Hand Screen U.S. Std Mesh #20, and the resulting mixture was blended using a bin blender to afford a granulated powder formulation for sublingual and/or buccal delivery.

An oral mucosal (e.g., sublingual and/or buccal) aspirin formulation was also prepared using a process comprising dry blending. Aspirin USP 80 mesh powder, tri-potassium citrate monohydrate, and aspartame 100 mesh powder were sieved using a Porta Sifter. The resulting mixture was blended with citric acid anhydrous powder and sodium lauryl sulfate using a V Blender, and the blended mixture was micronized using a jet mill micronizer (Sturtevant Micronizer) to afford a micronized aspirin dry blend. Aerosil 200 Pharma was sieved using Hand Screen U.S. Std Mesh #20, and the resulting sieved Aerosil 200 Pharma was blended with the micronized aspirin dry blend using a bin blender to afford a powder formulation for sublingual delivery.

In each formulation method (i.e., fluid bed granulation and dry blending), micronization of the aspirin together with additional excipients (e.g., tri-potassium citrate monohydrate, citric acid anhydrous, and sodium lauryl sulfate) significantly improved the uniform distribution of aspirin in the blend. The blend uniformity was maintained through subsequent process steps, as demonstrated in the blend uniformity data provided below.

Micronized aspirin pre-blend batch #1 (F22006-001) was prepared by air jet milling as described above with a batch size of 5.5 kg. To remove large lumps and prevent interference with the feed rate, individual ingredients were screened through US mesh #14. Formulation composition and process parameters/in-process data for pre-blend batch #1 are provided in Table 2 and Table 3, respectively.

TABLE 2
Formulation Composition

| Ingredients | Amount (g) |
|---|---|
| Aspirin USP 80 mesh Powder | 2604.6 |
| Citric Acid anhydrous, powder | 2056.2 |
| Tri-potassium citrate monohydrate | 78.6 |
| Vitasweet Aspartame 100 mesh | 520.8 |
| powder | |
| Sodium Lauryl Sulfate | 217.2 |

TABLE 3
Process Parameters & In-process data

| Process Parameters | Data | |
|---|---|---|
| Bin Blender (CML-1200/35 L) RPM | 20 | |
| Blending Duration | 3 | min |
| Powder feed rate/Feeder setting | 485/425 | |
| Grind air/Feed air pressure | 50/50 | PSIG |
| Milling duration | 20 | min |
| Bulk density pre-milled/post-milled | 0.69/0.33 | g/mL |

| | |
|---|---|
| LOD pre-milled/post-milled | NA/1.07% |

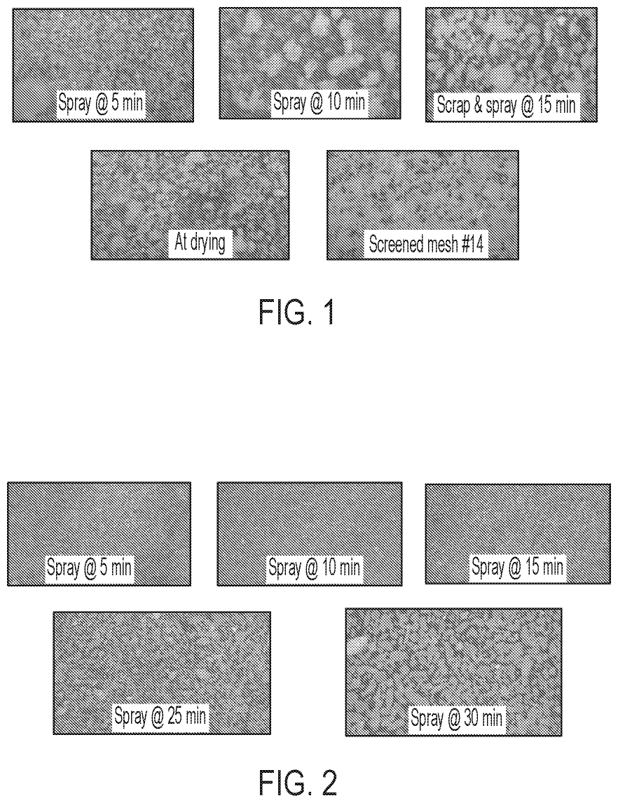

Fluid bed granulation batch #2 (F22006-002) was prepared using a top-spray process in a Glatt GPCG 3.1 fluid bed dryer with a batch size of 2.9 kg. Sodium lauryl sulfate (SLS) at 0.3% concentration in purified water USP was sprayed on micronized aspirin blend (batch #1) (batch F22006-001) during granulation. Microscopic images of samples taken at various spraying intervals are provided in FIG. 1. The resultant aspirin FB granules were blended with Aerosil 200 to obtain finished granular product. Formulation composition and process parameters/in-process data for fluid bed granulation batch #2 are provided in Table 4 and Table 5, respectively.

TABLE 4
Formulation Composition

| Ingredients | Amount (g) |
|---|---|
| Micronized aspirin Pre-Blend batch #1 | 2690.0 |
| Granulation Liquid | |
| Sodium Lauryl Sulfate | 0.8 |
| Purified Water USP | 266.7 |
| Blending | |
| Aspirin FB granules | 1912.9 |
| Aerosil 200 Pharma | 181.8 |

TABLE 5
Process Parameters & In-process data

| Process Parameters | Data | |
|---|---|---|
| Product Temperature T₀-T_{end} | 24.5-35.7° | C. |
| Air Flow T₀-T_{end} | 32-44 | CFM |
| Spray rate T₀-T_{end} | 14-15 | g/min |
| Spraying Duration | ~27 | min |
| Bin Blender (CML-10/10 L) RPM | 20 | |
| Blending Duration | 3 | min |
| Bulk density | 0.247 | g/mL |

| | |
|---|---|
| LOD | 0.62% |

Fluid bed granulation batch #3 (F22006-003) was prepared using a top-spray process in Glatt GPCG 3.1 fluid bed dryer with a batch size of 2.9 kg. SLS at 0.3% concentration in purified water USP was sprayed on micronized aspirin blend (batch #1) (batch F22006-001) during granulation. Microscopic images of samples taken at various spraying intervals are provided in FIG. 2. The resultant aspirin FB granules were blended with Aerosil 200 to obtain finished granular product. Formulation composition and process parameters/in-process data for fluid bed granulation batch #3 are provided in Table 6 and Table 7, respectively.

TABLE 6
Formulation Composition

| Ingredients | Amount (g) |
|---|---|
| Micronized aspirin Pre-Blend batch #1 | 2690.0 |
| Granulation Liquid | |
| Sodium Lauryl Sulfate | 0.8 |
| Purified Water USP | 266.7 |
| Blending | |
| Aspirin FB granules | 2615.0 |
| Aerosil 200 Pharma | 248.6 |

TABLE 7
Process Parameters & In-process data

| Process Parameters | Data | |
|---|---|---|
| Product Temperature T₀-T_{end} | 24.1-26.2° | C. |
| Air Flow T₀-T_{end} | 38-36 | CFM |
| Spray rate T₀-T_{end} | 8-8 | g/min |
| Spraying Duration | ~40 | min |
| Bin Blender (CML-1200/35 L) RPM | 18 | rpm |
| Blending Duration | 3 | min |
| Bulk density | 0.248 | g/mL |

| | |
|---|---|
| LOD | 0.54% |

Pre-blend granulation batch #4 (F22006-004) was prepared using an air jet milling process similar to pre-blend batch #1 (F22006-001) with a batch size of 2.7 kg. Dry blend granulation batch #5 (F22006-005) was prepared by blending pre-blend batch #4 in a CM 1200 bin blender with a batch size of 2.9 kg. Formulation composition and process parameters/in-process data for dry blend granulation batch #5 are provided in Table 8 and Table 9, respectively.

TABLE 8
Formulation Composition

| Ingredients | Amount (g) |
|---|---|
| Micronized aspirin Pre-Blend batch #4 | 2662.6 |
| Aerosil 200 Pharma | 253.1 |

TABLE 9
Process Parameters & In-process data

| Process Parameters | Data | |
|---|---|---|
| Bin Blender (CM-1200/35 L) RPM | 18 | rpm |
| Blending Duration | 3 | min |
| Bulk density | 0.205 | g/mL |

| | |
|---|---|
| LOD | 0.43% |

### Aspirin Content Assay

Batch nos. F22006-002, F22006-003, and F22006-005 were analyzed for aspirin content using standard methods known in the art. Four samples were collected from each batch using a 1.0 mm die, and the aspirin content for each batch was analyzed by HPLC (methods provided below).

### HPLC Methods and Parameters

Standard preparation: Approximately 25 mg USP aspirin was transferred into a 25 mL amber volumetric flask and dissolved in 60/40 0.2% phosphoric acid in USP water/acetonitrile diluent to volume. The resulting solution was mixed well to afford a stock solution, which was diluted to a concentration of about 0.1 mg/mL.

Sample preparation: Blend batch amounts equivalent to 200 mg aspirin were transferred into 100 mL volumetric flasks and dissolved in 0.2% phosphoric acid in 60/40 USP water/acetonitrile diluent to volume. The resulting solution was mixed well to afford a stock solution, which was diluted to a concentration of about 0.1 mg/mL. The sample solutions were immediately transferred into HPLC vials and maintained in the HPLC system at 4° C.

HPLC Instrument Details: Waters HPLC; Column: Hypersil GOLD aQ 4.6*250 mm, 5 micron; Column temperature: 30° C.; Sample temperature: 4° C.; Injection volume: 10 μL; Mobile Phase A: 0.2% phosphoric acid in USP water; Mobile Phase B: 0.2 phosphoric acid in ACN; Mobile phase gradient details: 100% A 0-15 min, 40/60 A/B 15-15.5 min, 100% B 15.5-19 min; Flow rate: 1.0 ml/min; Run time: 19 min; UV Detection Method: PDA, Wavelength: 215 nm

The aspirin content for each batch is summarized in Table 10, Table 11, and Table 12 respectively.

TABLE 10
Aspirin content of batch #F22006-002
Assay, % LC
Assay Summarized by Name
Label: Uf

| | | | | Sample | |
|---|---|---|---|---|---|
| | Sample Name | Vial | Inj | Weight, mg | Aspirin |
| 1 | Blend F22006-002_Assay-1 | 4 | 1 | 461.90 | 97.08 |
| 2 | Blend F22006-002_Assay-1 | 4 | 2 | 461.90 | 97.65 |
| 3 | Blend F22006-002_Assay-2 | 5 | 1 | 462.77 | 96.31 |
| | Blend F22006-002_Assay-2 | 5 | 2 | 462.77 | 96.85 |
| Mean | | | | | 97.0 |

TABLE 11
Aspirin content of batch #F22006-003
Assay, % LC
Assay Sunmmrized by Name
Label: U2

| | | | | Sample | |
|---|---|---|---|---|---|
| | Sample Name | Vial | Inj | Weight, mg | Aspirin |
| 1 | Blend F22006-003_Assay-1 | 5 | 1 | 463.00 | 98.69 |
| 2 | Blend F22006-003_Assay-1 | 5 | 2 | 463.00 | 99.58 |
| 3 | Blend F22006-003_Assay-2 | 6 | 1 | 461.24 | 99.69 |
| 4 | Blend F22006-003_Assay-2 | 6 | 2 | 461.24 | 99.44 |
| Mean | | | | | 99.4 |

TABLE 12
Aspirin content of batch #F22006-005
Assay, % LC
Assay Summarized by Name
Label: U3

| | | | | Sample | |
|---|---|---|---|---|---|
| | Sample Name | Vial | Inj | Weight, mg | Aspirin |
| 1 | Blend F22006-005_Assay-1 | 7 | 1 | 462.01 | 95.53 |
| 2 | Blend F22006-005_Assay-1 | 7 | 2 | 462.01 | 95.79 |
| 3 | Blend F22006-005_Assay-2 | 8 | 1 | 462.81 | 99.38 |
| 4 | Blend F22006-005_Assay-2 | 8 | 2 | 462.81 | 99.57 |
| Mean | | | | | 97.6 |

### Blend Uniformity

Batches #F22006-002, F22006-003, and F22006-005 were analyzed for blend/content uniformity. Ten blend uniformity (BU) samples were collected using a 1.0 mm die. Blend samples were prepared at concentrations of approximately 0.1 mg/mL. The samples were analyzed by HPLC using the same methods described above for the aspirin content assay.

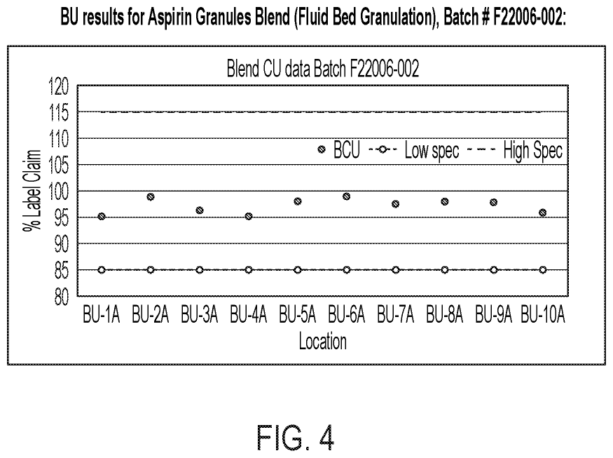

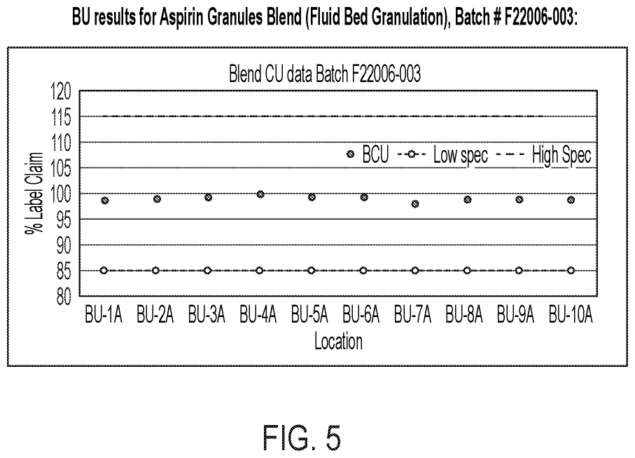

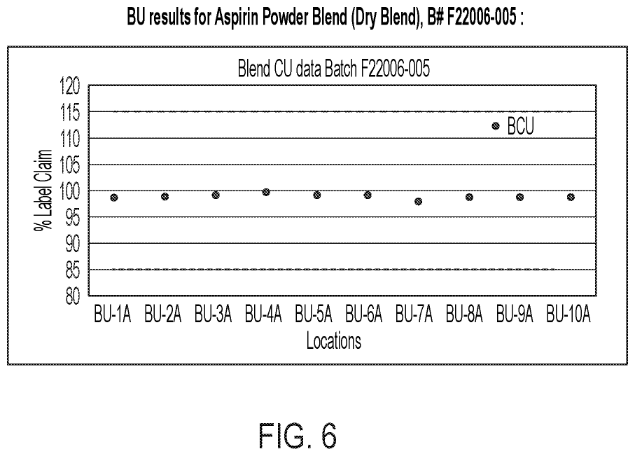

The blend uniformity for each batch is summarized in Table 13 and FIGS. 4-6.

TABLE 13
Blend uniformity of batch nos. F22006-
002, F22006-003, and F22006-005

| Sample | % Mean (N = 10) | SD (%) | Max/Min |
|---|---|---|---|
| F22006-002 | 97.1 | 1.4 | 99.0/95.1 |
| F22006-003 | 99.0 | 0.5 | 100.0/98.0 |
| F22006-005 | 98.3 | 0.7 | 99.4/97.5 |

### Example 2. Determination of pH for Oral Mucosal (e.g., Sublingual and/or Buccal) Aspirin Formulations

The pH values of aspirin API and batch nos. F22006-001, F22006-004, F22006-002, F22006-003, and F22006-005 were determined using standard protocols as described in the United States Pharmacopoeia, Chapter <791>. USP water was added to each aspirin API and pre-blend/blend batches, and the mixture was stirred for 15 minutes. The pH of the mixture was measured using a Mettler Toledo pH meter.

Experimental parameters and pH values are given in Table 14.

TABLE 14
Determination of pH for aspirin samples

| | | | Dilution | |
|---|---|---|---|---|
| | | Weight of | in USP | |
| Sample | Batch # | Sample (mg) | Water (mL) | pH |
| Aspirin API | 22297 | 1861.9 | 5.0 | 2.62 |
| Aspirin Pre-Blend | F22006-001 | 1864.1 | 10.0 | 2.36 |
| (Fluid Bed | | | | |
| Granulation) | | | | |
| Aspirin Pre-Blend | F22006-004 | 1866.3 | 10.0 | 2.36 |
| (Dry Blend) | | | | |
| Aspirin Granules | F22006-002 | 1866.1 | 10.0 | 2.31 |
| Blend (Fluid Bed | | | | |
| Granulation) | | | | |
| Aspirin Granules | F22006-003 | 1864.8 | 10.0 | 2.32 |
| Blend (Fluid Bed | | | | |
| Granulation) | | | | |
| Aspirin Powder | F22006-005 | 1866.7 | 10.0 | 2.30 |
| Blend (Dry Blend) | | | | |

### Example 3. Dissolution of Oral Mucosal (e.g., Sublingual and/or Buccal) Aspirin Formulations

The example describes studies that were performed to investigate the dissolution rate and solubility of batch #F22006-003 (fluid bed granulation) and F22006-005 (dry blend).

### Dissolution Rate

Batch nos. F22006-003 and F22006-005 were evaluated for their dissolution profiles. Samples were subjected to dissolution conditions in a Distek dissolution apparatus and were then analyzed by HPLC (dissolution and HPLC methods and parameters provided below):

### Dissolution Apparatus Conditions and Parameters

apparatus: Distek dissolution bath coupled to Waters HPLC
dissolution medium: pH 6.8 potassium phosphate buffer, 500 mL
apparatus paddle speed: 50 rpm

$$n = 3$$

sample collection time points: 10 min, 30 min
sample collection volume: 9 mL
Sample size: 1 dose equivalent (~186.6 mg of formulation)

### HPLC Methods and Parameters

Standard preparation: Approximately 20 mg USP aspirin diluted to volume with pH 6.8 phosphate buffer in 100 mL amber volumetric flask (the API was sonicated for about 5 minutes in about 70 mL pH 6.8 phosphate buffer first). The resulting solution had a concentration of about 0.16 mg/mL aspirin API.

HPLC Instrument Details: Waters HPLC; Column: Hypersil GOLD aQ 4.6*250 mm, 5 micron; Column temperature: 30° C.; Sample temperature: 4° C.; Injection volume: 10 μL; Mobile Phase A: 0.2% phosphoric acid in USP water; Mobile Phase B: 0.2 phosphoric acid in ACN; Mobile phase gradient details: 100% A 0-15 min, 40/60 A/B 15-15.5 min, 100% B 15.5-19 min; Flow rate: 1.0 ml/min; Run time: 19 min; UV Detection Method: PDA, Wavelength: 215 nm

Dissolution rate data are provided in Table 15. The results were calculated using single Aspirin peak area and combined Aspirin+Salicylic Acid peaks area.

TABLE 15
Dissolution of formulation batches F22006-003 and F22006-005

| | | F22006- | | | |
|---|---|---|---|---|---|
| | | 003 | | | |
| | | Mean % Released | | | |
| | | | | Calculated for | |
| | | Calculated for single | | combined Aspirin + | |
| | | Aspirin peak | | Salicylic Acid peaks | |
| | Active, | 10 | 30 | 10 | 30 |
| Potency | mg | minutes | minutes | minutes | minutes |
| 0.4341 | 81 | 88.7 | 97.2 | 89.2 | 99.4 |
| | | F22006- | | | |
| | | 005 | | | |
| | | Mean % Released | | | |
| | | | | Calculated for | |
| | | Calculated for single | | combined Aspirin + | |
| | | Aspirin peak | | Salicylic Acid peaks | |
| | Active, | 10 | 30 | 10 | 30 |
| Potency | mg | minutes | minutes | minutes | minutes |
| 0.4341 | 81 | 88.4 | 97.6 | 88.9 | 99.8 |

### Example 4: Particle Size of Blend Batch Nos. 2, 3, and 5

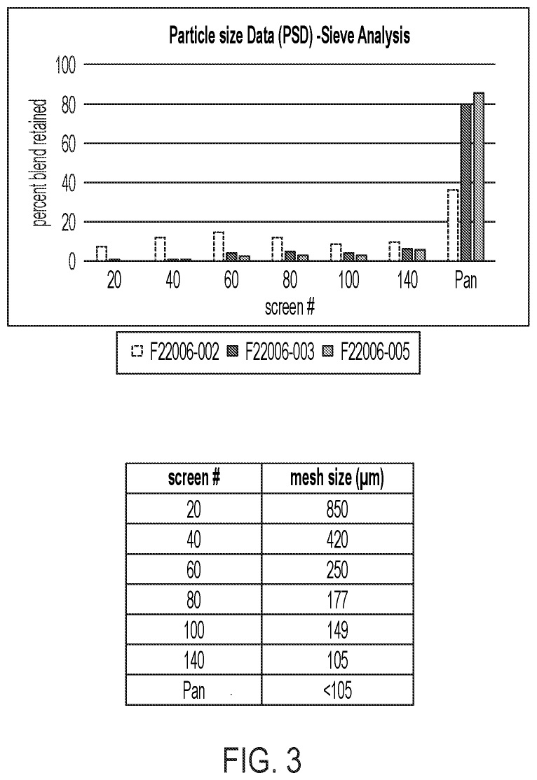

The particle size distribution of batch nos. F22006-002, F22006-003, and F22006-005 was determined using a RO-TAP sieve shaker. Each sieve was weighed before sample analysis. Approximately 50 g of each batch no. was added to the top sieve, and samples were shaken. The percent retained on each sieve is given in Table 16 and FIG. 3.

TABLE 16
Percent blend batches retained

| | | % Retained | | |
|---|---|---|---|---|
| Screen # | Mesh size (μm) | F22006-002 | F22006-003 | F22006-005 |
| 20 | 850 | 7.2 | 0.2 | 0.0 |
| 40 | 420 | 11.7 | 0.6 | 0.4 |
| 60 | 250 | 14.7 | 4.1 | 2.2 |
| 80 | 177 | 11.7 | 4.9 | 2.8 |
| 100 | 149 | 8.5 | 4.1 | 2.8 |
| 140 | 105 | 9.7 | 6.4 | 5.6 |
| Pan | <105 | 36.4 | 79.7 | 86.1 |

Approximately 80% of the blend was smaller than 105 microns. However, some agglomeration was observed during the first batch processing (F22006-002) that resulted in larger particle size distribution compared to batches F22006-003 and F22006-005.

### Example 5: Assay and Dissolution Studies after One Month of Storage

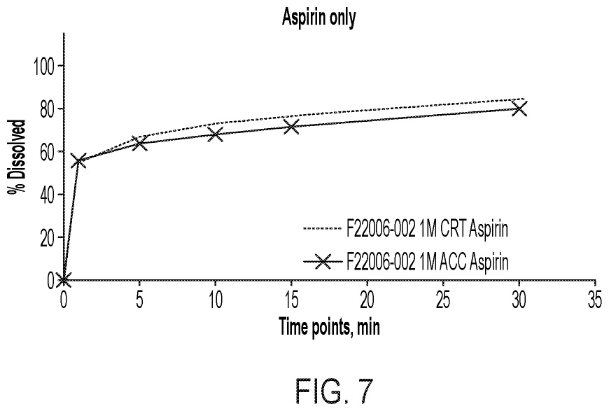

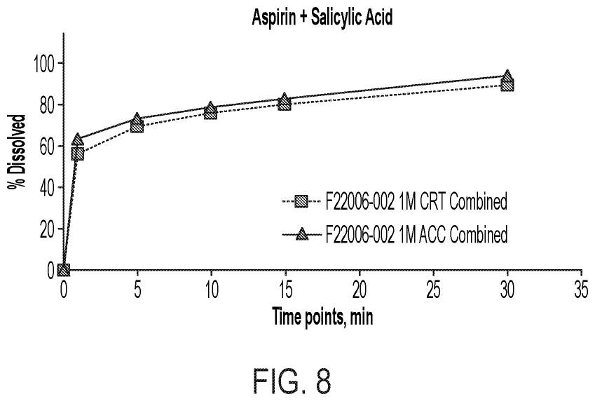

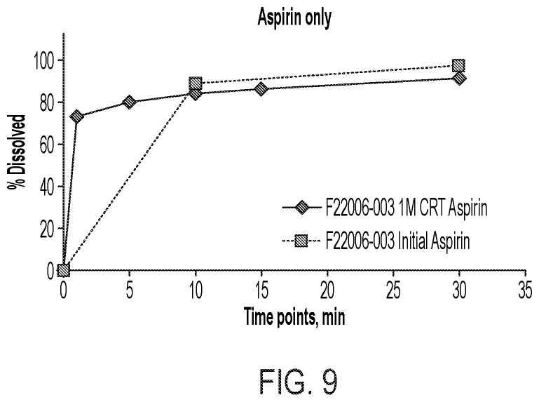

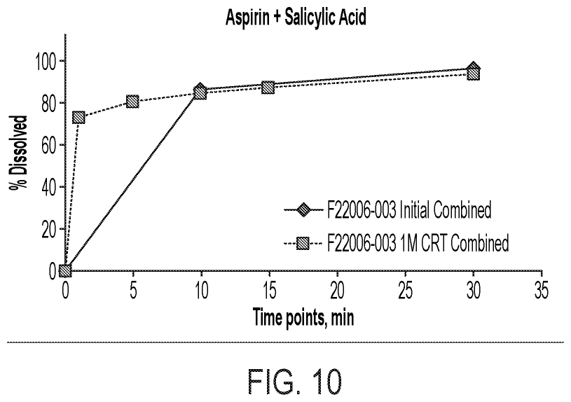

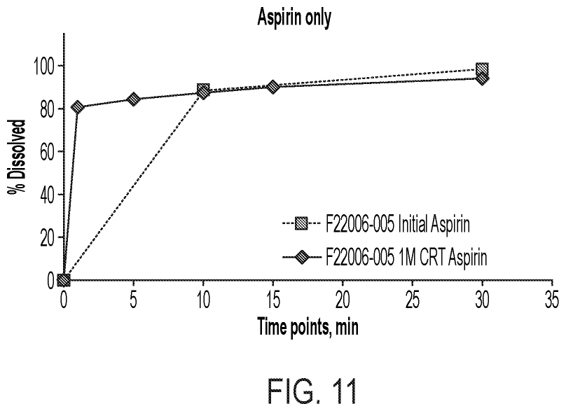

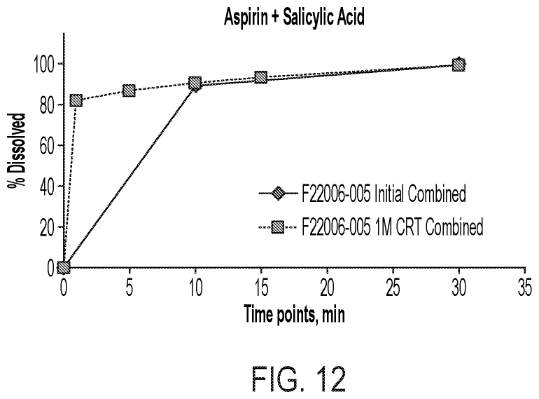

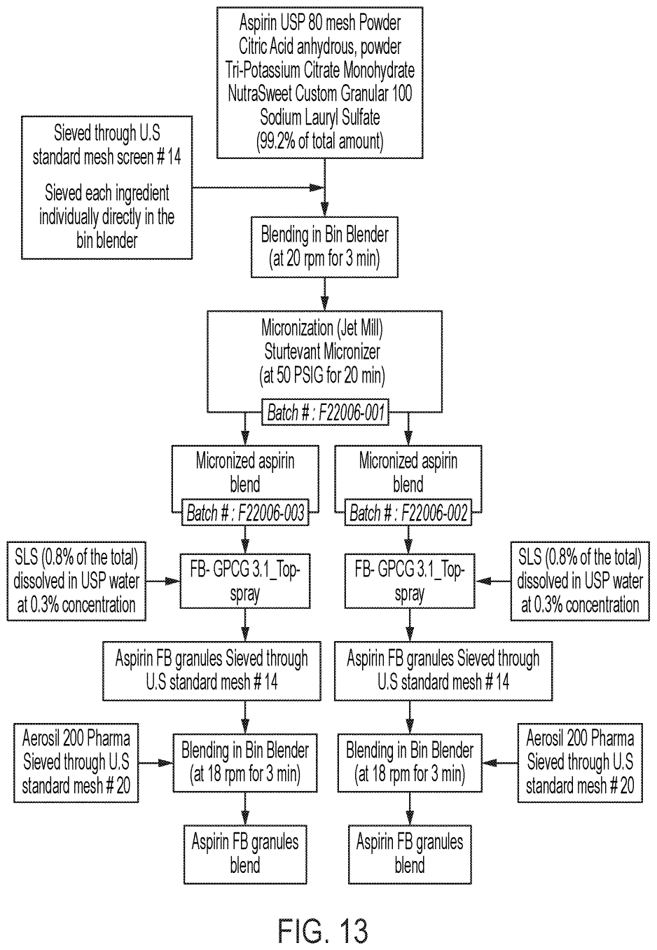

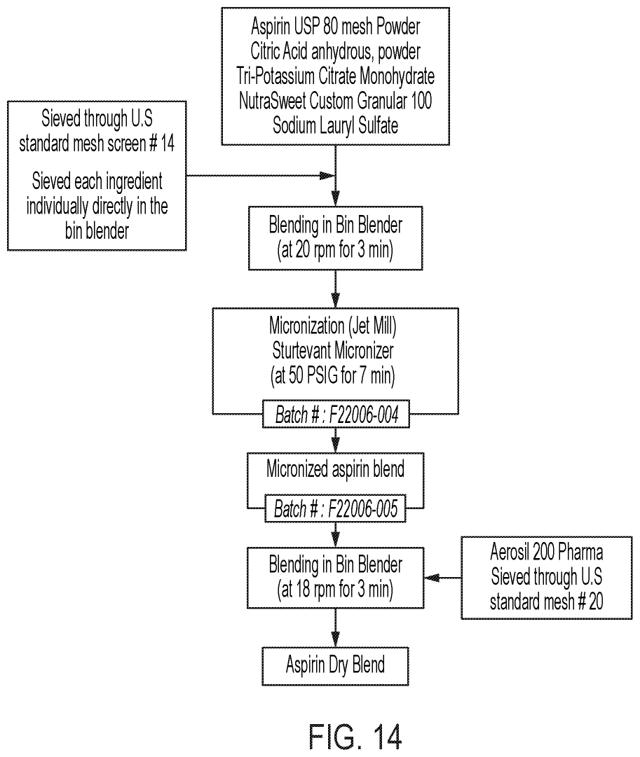

Formulation batches F22002-002, F22006-003, and F22006-005 were analyzed for their aspirin/impurity content and dissolution profile after one month of stable storage using the same methods described above. The formulation batches were stored in either controlled room temperature (CRT) (25° C./60% relative humidity) or accelerated temperature/humidity (ACC) (40° C./75% relative humidity). The results are provided in Tables 17-22 and FIGS. 7-12.

TABLE 17
Assay data of batch F22006-002 after one month of storage in CRT or ACC

| | | | Salicylic | % of Total | | Salicylic | % of Total |
|---|---|---|---|---|---|---|---|
| | | | acid | Unknown | | acid | Unknown |
| | | Assay % | content % | Impurity | Assay, % | content % | Impurity |
| Lot No. | Description | (1M) | (1M) | (1M) | (T = 0) | (T = 0) | (T = 0) |
| F22006-002- | FB | 98.7% | 0.6 | 0.31 | 97.0% | 0.04 | Not |
| 1M-CRT | granulation | | | | | | Detected |
| F22006-002- | FB | 92.7% | 6.3 | 5.12 | | | |
| 1M-ACC | granulation | | | | | | |

TABLE 18
Dissolution of batch F22006-002 after
one month of storage in CRT or ACC

Time point (min)

| Batch #/Condition | 0 | 1 | 5 | 10 | 15 | 30 |
|---|---|---|---|---|---|---|
| F22006-002/1M | 0 | 54 | 67 | 73 | 77 | 84 |
| CRT Aspirin | | | | | | |
| F22006-002/1M | 0 | 56 | 69 | 76 | 80 | 89 |
| CRT Combined | | | | | | |
| F22006-002/1M | 0 | 56 | 64 | 68 | 71 | 80 |
| ACC Aspirin | | | | | | |
| F22006-002/1M | 0 | 63 | 73 | 78 | 83 | 94 |
| ACC Combined | | | | | | |

TABLE 19
Assay data of batch F22006-003 after one month of storage in CRT or ACC

| | | | Salicylic | % of Total | | Salicylic | % of Total |
|---|---|---|---|---|---|---|---|
| | | | acid | Unknown | | acid | Unknown |
| | | Assay % | content % | Impurity | Assay, % | content % | Impurity |
| Lot No. | Description | (1M) | (1M) | (1M) | (T = 0) | (T = 0) | (T = 0) |
| F22006-003- | FB | 99.8% | 0.7 | 0.37 | 99.4% | 0.08 | Not |
| 1M-CRT | granulation | | | | | | Detected |
| F22006-003- | FB | 93.2% | 6.9 | 5.76 | | | |
| 1M-ACC | granulation | | | | | | |

TABLE 20
Dissolution of batch F22006-003 after
one month of storage in CRT or ACC

| | Time Point (min) | | | | | |
|---|---|---|---|---|---|---|
| Batch #/Condition | 0 | 1 | 5 | 10 | 15 | 30 |
| F22006-003/Initial | 0 | N/A | N/A | 89 | N/A | 97 |
| Aspirin | | | | | | |
| F22006-003/Initial | 0 | N/A | N/A | 89 | N/A | 99 |
| Combined | | | | | | |
| F22006-003/1M | 0 | 73 | 81 | 84 | 87 | 92 |
| CRT Aspirin | | | | | | |
| F22006-003/1M | 0 | 75 | 83 | 87 | 90 | 97 |
| CRT Combined | | | | | | |
| F22006-003/1M | N/A | N/A | N/A | N/A | N/A | N/A |
| ACC Aspirin | | | | | | |
| F22006-003/1M | N/A | N/A | N/A | N/A | N/A | N/A |
| ACC Combined | | | | | | |

TABLE 21
Assay data of batch F22006-005 after one month of storage in CRT or ACC

| | | | Salicylic | % of Total | | Salicylic | % of Total |
|---|---|---|---|---|---|---|---|
| | | | acid | Unknown | | acid | Unknown |
| | | Assay % | content % | Impurity | Assay, % | content % | Impurity |
| Lot No. | Description | (1M) | (1M) | (1M) | (T = 0) | (T = 0) | (T = 0) |
| F22006-005- | Dry blend | 100.9% | 0.6 | 0.35 | 97.6% | 0.08 | Not |
| 1M-CRT | Powder | | | | | | Detected |
| F22006-005- | Dry blend | 94.5% | 6.5 | 5.89 | | | |
| 1M-ACC | Powder | | | | | | |

TABLE 22
Dissolution of batch F22006-005 after
one month of storage in CRT or ACC

| | Time Point (min) | | | | | |
|---|---|---|---|---|---|---|
| Batch #/Condition | 0 | 1 | 5 | 10 | 15 | 30 |
| F22006-005/Initial | 0 | N/A | N/A | 88 | N/A | 98 |
| Aspirin | | | | | | |
| F22006-005/Initial | 0 | N/A | N/A | 89 | N/A | 100 |
| Combined | | | | | | |
| F22006-005/1M | 0 | 80 | 84 | 87 | 90 | 94 |
| CRT Aspirin | | | | | | |
| F22006-005/1M | 0 | 82 | 87 | 91 | 93 | 99 |
| CRT Combined | | | | | | |
| F22006-005/1M | N/A | N/A | N/A | N/A | N/A | N/A |
| ACC Aspirin | | | | | | |
| F22006-005/1M | N/A | N/A | N/A | N/A | N/A | N/A |
| ACC Combined | | | | | | |

### Example 6. Stability of Batch F22006-005 During First 6 Months of Storage

Formulation batches F2006-002, F22006-003, and F22006-005 were analyzed for their aspirin content (assay), moisture, and degradation after one month, three months, and six months of storage. The formulation batches were stored in controlled room temperature (CRT) (25° C./60% relative humidity). Aspirin assay/degradation was analyzed by HPLC. A reverse-phase HPLC method using Hypersil GOLD aQ, 4.6×250 mm, 5 μm column for a gradient elution with mobile phase A composed of 0.2% Phosphoric Acid in USP Water and mobile phase B composed of 0.2% Phosphoric Acid in ACN at a wavelength of 215 nm was utilized. Aspirin and Salicylic Acid standards and samples were prepared in 60:40 USP Water: ACN, v/v. Column temperature was 30° C., and sample temperature in autosampler was 4° C. The retention time was about 12 minutes for Aspirin and about 13.5 minutes for Salicylic Acid. Moisture was determined by Karl Fischer titration analysis.

The results are provided in Tables 23-25.

TABLE 23
Stability, moisture content, and degradation
of batch F22006-002 over time

| | 0 (Initial) | | 1 Month | | 3 Months | | 6 Months | |
|---|---|---|---|---|---|---|---|---|
| Potency (%) | 43.4% | | 43.4% | | 43.4% | | 43.4% | |
| Appearance | White to | | White to | | White to | | White to | |
| | off-white | | off-white | | off-white | | off-white | |
| | granules | | granules | | granules | | granules | |
| Assay | 97.0% | | 98.7% | | 97.5% | | 95.1% | |
| Moisture (KF) | N/A | | 0.6% | | 0.4% | | 0.7% | |
| Degradation | RRT | % | RRT | % | RRT | % | RRT | % |
| Any Unspecified | | | 0.71 | 0.11 | 0.70 | 0.14 | 0.70 | 0.16 |
| Individual | | | 0.76 | 0.05 | 0.75 | 0.05 | 0.98 | 0.45 |
| | | | 0.93 | 0.15 | 0.91 | 0.03 | | |
| | | | | | 0.97 | 0.33 | | |
| | N/A | | | | | | | |

| | | | |
|---|---|---|---|
| Salicylic Acid | 0.56% | 0.95% | 1.35% |
| Total | 0.87% | 1.5% | 2.0% |

TABLE 24
Stability, moisture content, and degradation
of batch F22006-003 over time

| | 0 (Initial) | | 1 Month | | 3 Months | | 6 Months | |
|---|---|---|---|---|---|---|---|---|
| Potency (%) | 43.4% | | 43.4% | | 43.4% | | 43.4% | |
| Appearance | White to | | White to | | White to | | White to | |
| | off-white | | off-white | | off-white | | off-white | |
| | granules | | granules | | granules | | granules | |
| Assay | 99.4% | | 99.8% | | 97.5% | | 96.6% | |
| Moisture (KF) | N/A | | 0.5% | | 0.4% | | 0.6% | |
| Degradation | RRT | % | RRT | % | RRT | % | RRT | % |
| Any Unspecified | | | 0.71 | 0.08 | 0.70 | 0.09 | 0.70 | 0.11 |
| Individual | | | 0.76 | 0.05 | 0.75 | 0.05 | 0.98 | 0.61 |
| | | | 0.93 | 0.24 | 0.97 | 0.46 | | |
| | N/A | | | | | | | |

| | | | |
|---|---|---|---|
| Salicylic Acid | 0.65% | 1.0% | 1.42% |
| Total | 1.02% | 1.6% | 2.1% |

TABLE 25
Stability, moisture content, and degradation
of batch F22006-005 over time

| | 0 (Initial) | | 1 Month | | 3 Months | | 6 Months | |
|---|---|---|---|---|---|---|---|---|
| Potency (%) | 43.4% | | 43.4% | | 43.4% | | 43.4% | |
| Appearance | White to | | White to | | White to | | White to | |
| | off-white | | off-white | | off-white | | off-white | |
| | granules | | granules | | granules | | granules | |
| Assay | 97.6% | | 100.9% | | 98.3% | | 99.2% | |
| Moisture (KF) | N/A | | 0.4% | | 0.4% | | 0.6% | |
| Degradation | RRT | % | RRT | % | RRT | % | RRT | % |
| Any Unspecified | | | 0.76 | 0.06 | 0.70 | 0.03 | 0.75 | 0.05 |
| Individual | | | 0.97 | 0.29 | 0.75 | 0.05 | 0.98 | 0.53 |
| | | | | | 0.97 | 0.46 | | |
| | N/A | | | | | | | |

| | | | |
|---|---|---|---|
| Salicylic Acid | 0.58% | 0.83% | 1.01% |
| Total | 0.93% | 1.37% | 1.6% |

### Example 7. A Phase 1 Pharmacokinetic/Pharmacodynamic Study to Compare Sublingual Aspirin Formulations with Standard Aspirin Formulations

This example describes a Phase 1 clinical study to compare the pharmacokinetic and pharmacodynamic characteristics of a sublingually-administered oral-mucosal formulation of buffered aspirin powder and standard orally administered aspirin formulations.

### Dosing Scheme

| Group 1 | Group 2 (control) |
|---|---|
| buffered sublingual aspirin | oral aspirin tablet (81 mg aspirin) |
| powder (186.8 mg) (Instaprin) | (Bayer) |

### Oral-Mucosal Formulation of Buffered Aspirin Powder Composition

| Ingredients | Amount (mg) |
|---|---|
| Aspirin USP 80 mesh Powder | 81.00 |
| Citric Acid anhydrous, | 63.95 |
| powder | |
| Tri-potassium citrate | 2.44 |
| monohydrate | |
| Aspartame 100 mesh powder | 16.20 |
| Sodium Lauryl Sulfate | 6.81 |
| Aerosil 200 Pharma | 16.20 |
| Total | 186.60 |

Participants provide written informed consent to participate in the study after Institutional Review Board (IRB) approval review of the protocol and then are randomized into two groups. Group 1 receives sublingual aspirin powder, and the control group (Group 2) receives Bayer aspirin tablets. Venous blood samples (5.0 ml) are collected using an in-dwelling cannula into monovettes containing 50 mg solid lithium fluoride before acetylsalicylic acid (ASA) dosing and at scheduled time intervals after ASA dosing. Plasma is separated immediately after venous blood sample collection by centrifugation and stored at −70° C. Plasma concentration of ASA and salicylic acid is measured using reverse phase high performance liquid chromatography (Aqua C18 R columns).

The primary PK parameters are:
Area under the plasma concentration versus time curve from zero to infinity after study drug dosing divided by drug dose (AUC/D)
Cₘₐₓ divided by drug dose (Cₘₐₓ/D)
Tₘₐₓ, i.e., time to reach the maximum drug concentration
Bioavailability

### Arms and Interventions

Healthy adult volunteers given sublingual aspirin (186 mg powder containing 81 mg aspirin) or oral aspirin tablet (81 mg aspirin). Single dose. Sublingual aspirin powder (Instaprin) and conventional aspirin (81 mg) manufactured by Bayer.

### Primary Outcome Measure

Plasma acetylsalicylic acid concentration versus time. Time frame: pre-dose (baseline), and 5 minutes, 30 minutes, 45 minutes, 60 minutes, 1.5 hours, 2 hours, 3 hours, 6 hours, 8 hours, and 24 hours post dose.

### Secondary Outcome Measures

Plasma thromboxane B2 concentration, pre-dose and up to 24 hours post dose
Platelet aggregation: Analyze for group difference between sublingual and oral aspirin treated groups

### Exclusion Criteria

The subject has received treatment with aspirin within 30 days
The subject is currently enrolled in another clinical study
The subject has history of chronic disease including kidney disease, cardiovascular disease, diabetes, gastrointestinal disease, and cancer
The subject has sensitivity to aspirin, aspartame, sodium lauryl sulfate (SDS), and/or potassium citrate
The subject has hepatitis, HIV, or COVID-19
The subject has Sjorgren disease, dry mouth, or mouth sores

1. A pharmaceutical formulation for oral mucosal (e.g., sublingual and/or buccal) administration comprising:
70 mg to 90 mg micronized aspirin;
a buffer;
a surfactant; and
one or more pharmaceutically acceptable excipients,
wherein the pH of a solution of the formulation dissolved in 1 mL distilled water is less than 3.

2. The pharmaceutical formulation of claim 1, wherein the formulation comprises granules comprising an intragranular fraction, wherein the intragranular fraction comprises the micronized aspirin.

3. The pharmaceutical formulation of claim 2, wherein the intragranular fraction further comprises the buffer.

4. The pharmaceutical formulation of claim 1, wherein the formulation does not comprise granules.

5. The pharmaceutical formulation of any one of claims 1-4, wherein the pH of a solution of the formulation dissolved in 1 ml distilled water is less than 2.8.

6. The pharmaceutical formulation of any one of claims 1-5, wherein the pH of a solution of the formulation dissolved in 1 mL distilled water is less than 2.5.

7. The pharmaceutical formulation of any one of claims 1-6, wherein the pH of a solution of the formulation dissolved in 1 mL distilled water is less than 2.2.

8. The pharmaceutical formulation of claim 1, wherein the pH of a solution of the formulation dissolved in 1 mL distilled water is 2 to 2.5.

9. The pharmaceutical formulation of claim 1, wherein the pH of a solution of the formulation dissolved in 1 mL distilled water is 1.8 to 2.5.

10. The pharmaceutical formulation of any one of claims 1-9, wherein the buffer concentration of the formulation is at least 100 mM when dissolved in 1 mL of distilled water.

11. The pharmaceutical formulation of any one of claims 1-10, wherein the amount of micronized aspirin in the formulation is 75 mg to 85 mg.

12. The pharmaceutical formulation of any one of claims 1-11, wherein the amount of micronized aspirin in the formulation is 81 mg.

13. The pharmaceutical formulation of any one of claims 1-12, wherein the buffer comprises citric acid, or a hydrate thereof, and a citrate salt, or a hydrate thereof.

14. The pharmaceutical formulation of any one of claims 1-12, wherein the buffer consists of citric acid, or a hydrate thereof, and a citrate salt, or a hydrate thereof.

15. The pharmaceutical formulation of claim 13 or 14, wherein the citric acid or hydrate thereof is citric acid anhydrous.

16. The pharmaceutical formulation of any one of claims 13-15, wherein the citrate salt or hydrate thereof is tri-potassium citrate monohydrate.

17. The pharmaceutical formulation of any one of claims 1-16, wherein the surfactant is an anionic surfactant.

18. The pharmaceutical formulation of any one of claims 1-17, wherein the surfactant is selected from the group consisting of sodium lauryl sulfate, ammonium lauryl sulfate, potassium lauryl sulfonate, potassium lauryl phosphate, sodium deoxycholate, and potassium deoxycholate.

19. The pharmaceutical formulation of any one of claims 1-18, wherein the surfactant is sodium lauryl sulfate.

20. A pharmaceutical formulation for oral mucosal (e.g., sublingual and/or buccal) administration comprising:
70 mg to 90 mg micronized aspirin;
56 mg to 75 mg citric acid anhydrous;
1 mg to 5 mg tri-potassium citrate monohydrate;
1 mg to 10 mg sodium lauryl sulfate; and
one or more pharmaceutically acceptable excipients.

21. A pharmaceutical formulation for oral mucosal (e.g., sublingual and/or buccal) administration comprising:
81 mg micronized aspirin;
64 mg citric acid anhydrous;
2.4 mg tri-potassium citrate monohydrate;
6.8 mg sodium lauryl sulfate; and
one or more pharmaceutically acceptable excipients.

22. The pharmaceutical formulation of any one of claims 1-21, wherein at least 85% of the micronized aspirin is released within 10 minutes using a paddle dissolution apparatus.

23. The pharmaceutical formulation of any one of claims 1-22, wherein at least 95% of the micronized aspirin is released within 30 minutes using a paddle dissolution apparatus.

24. The pharmaceutical formulation of any one of claims 1-23, wherein the formulation comprises an anti-settling and/or anti-sagging agent.

25. The pharmaceutical formulation of claim 24, wherein the anti-settling and/or anti-sagging agent is fumed silica.

26. The pharmaceutical formulation of any one of claims 1-25, wherein the formulation comprises one or more of: aspartame, sodium lauryl sulfate, and fumed silica.

27. The pharmaceutical formulation of any one of claims 1-25, wherein the formulation comprises each of aspartame, sodium lauryl sulfate, and fumed silica.

28. The pharmaceutical formulation of any one of claims 1-27, wherein the formulation is a solid dosage form.

29. The pharmaceutical formulation of any one of claims 1-28, wherein the formulation is in powder form.

30. A kit for suitable storage of an oral-mucosal aspirin formulation comprising:
a pouch suitable for packaging the formulation of any one of claims 1-28; and
the formulation of any one of claims 1-29.

31. A method of treating a disease, disorder, or condition in a subject in need thereof, the method comprising administering to the subject a formulation of any one of claims 1-29.

32. The method of claim 31, wherein the disease, disorder, or condition is selected from pain, fever, and swelling.
